(19) United States
(12) Patent Application Publication
(10) Pub. No.: US 2022/0117649 A1
(43) Pub. Date: Apr. 21, 2022

(54) Apparatus for Attachment of a Light Receiving Device to a Surgical Instrument

(71) Applicant: Erbe Elektromedizin GmbH, Tuebingen (DE)

(72) Inventors: Markus Reiterer, Loipersbach (AT); Achim Brodbeck, Metzingen (DE); Klaus Fischer, Nagold (DE); Marc Mueller, Tuebingen (DE)

(21) Appl. No.: 17/500,317

(22) Filed: Oct. 13, 2021

(30) Foreign Application Priority Data
Oct. 19, 2020 (EP) 20202484.0

(51) Int. Cl. A61B 18/14

(52) U.S. Cl. CPC A61B 18/1402; A61B 2017/00057

(57) ABSTRACT

An apparatus (10) serves for attachment of a light receiving device (24) for light analysis on an instrument (11) or an instrument component (12) during surgery by the surgery user of a surgical instrument (11) or their assistant. The apparatus (10) is preferably configured to releasably attach the light receiving device (24) on the instrument (11) or the instrument component (12). The apparatus (10) can comprise a light receiving device (24) and an electrode (15) fixed relative thereto. The electrode (15) can alternatively also be part of the instrument (11). The apparatus (10) can be releasably attachable to the instrument (11) or the instrument component (12) and can form an adapter for attachment of the light receiving device (24) on the instrument (11) or the instrument component (12) or can also be configured as part of the instrument (10).

### RELATED APPLICATION(S)

This application claims the benefit of European Patent Application No. 20202484.0, filed Oct. 19, 2020, the contents of which are incorporated herein by reference as if fully rewritten herein.

### TECHNICAL FIELD

The present disclosure refers to the field of analysis of light appearances, e.g. sparks, that can be created by radio frequency surgical instruments.

### BACKGROUND

In applications EP 3 284 430 A1 and EP 2 659 846 A1 an instrument is disclosed having an electrode and light receiving devices that can be orientated in different orientations facing the electrode.

US 2009/0088772 A1 shows an instrument that can be carried by a robot that has a distal working section and an optical fiber that can transmit light from the distal end of the instrument for analysis of light.

According to EP 2 815 713 A1 an optical fiber is arranged in an electrode.

In order to analyze light of a light appearance, particularly a radio frequency spark, created due to a radio frequency surgical intervention in the clinical use, optical fibers are used as light receiving device that is connected with an analysis device for determination of light features and can output information with regard to the biological tissue during surgery. The optical fiber can be contaminated by blood, tissue liquid, fume (aerosol), due to tissue contact of the distal end of the optical fiber as well as contact of the optical fiber with the tissue, due to flaking of tissue and spraying away of tissue particles and can remarkably reduce or even completely block the transmission of light to the operation unit in this manner. Also by sticking of tissue to the distal end of the analysis device that can indirectly result in shading of the optical fiber, reduction of the transmission of light is possible. A contamination of the optical fiber can distort the light signal in its intensity, because the contamination can absorb light with different amounts. Also a condensation of liquid on the fiber can temporarily modify the transmission. Due to the contamination, the transmission can also be modified depending on the wavelength, which can result in a distortion of the analysis result. Due to a high input of energy (e.g. 300 Watt) during radio frequency surgery, very high temperatures (e.g. higher than 300° C.) can be created at the tip of the radio frequency electrode. Also high temperatures can thermally damage the optical fiber, whereby the transmission of light can be reduced or completely impeded as well.

One object of the present invention is to provide an improved concept for a light receiving device.

### SUMMARY

This object is solved by means of an apparatus and by means of a method as described herein.

The inventive apparatus in one form is configured to be attached preferably releasably and particularly releasably in a non-destructive manner with a surgical instrument, particularly an electrosurgical instrument, or to an instrument component of the surgical instrument. The apparatus comprises a light receiving device (particularly optical fiber) for light analysis, particularly by a medically educated user, e.g. a physician. The attachment can be carried out during surgery (inside the operation room), e.g. after a surgery step has been carried out by means of the instrument. The apparatus allows the capture or reception of light by means of the light receiving device (particularly optical fiber) that is created during use of the surgical instrument. The received light can be transmitted by means of the light receiving device for the purpose of light analysis. The light receiving device of the apparatus can also capture light suitable for the light analysis during use of the instrument.

By means of the apparatus the light receiving device can, for example, be attached to a handle of the instrument, only if the extraction of a tissue information by means of light analysis is clinically desired. With preferred embodiments for releasable attachment the light receiving device can be easily exchanged by the user by a new, clean light receiving device. Preferably the apparatus does not have to be destroyed for this purpose. The connection of the light receiving device with the surgical instrument by means of the apparatus is preferably possible without tools. The apparatus can be preferably plugged on the instrument or, at least partly, pushed into the instrument and/or the instrument can be preferably plugged in the apparatus or at least partly pushed in the apparatus and/or the apparatus can be plugged in the instrument or at least partly pushed into the instrument.

According to another aspect of the invention, a method is disclosed that comprises the attachment of a light receiving device to a surgical instrument or an instrument component of the surgical instrument by the surgeon or his assistant during surgery. The surgeon or his assistant combines the light receiving device with the instrument or the instrument component to form an operable unit by means of which in embodiments a radio frequency surgical intervention can be particularly carried out under supervision by means of an optical emission spectroscopy. The surgeon or his assistant can use an inventive apparatus for this purpose, for example, as disclosed herein.

The inventive apparatus can be embodied, for example, by at least one or multiple features indicated in the following.

The instrument component of the surgical instrument can be, for example, a component or a unit of the instrument.

Preferably the apparatus is configured for attachment of a light receiving device (particularly optical fiber) for light analysis to an operable surgical instrument during surgery (in the operation room), e.g. after a surgery step has been carried out with the instrument, by a medically educated user, e.g. a physician. The instrument is in embodiments also already operable without the apparatus and the light receiving device, when the light receiving device is attached to the instrument by means of the apparatus. For example, the instrument component can already be connected with an electrode prior to the connection with the apparatus, such that the instrument component forms an operable radio frequency surgical instrument together with the electrode.

As an alternative or in addition, the apparatus is preferably configured to complete the instrument component during attachment of the light receiving device on the instrument component to form an operable instrument. For example, the apparatus can be configured to support an electrode.

Particularly if the apparatus comprises an electrode and a light receiving device, but also in other embodiments, it is preferred, if the apparatus is configured to define the spatial relative position (relative location and/or relative orientation) of the electrode and the light receiving device relative to one another. In doing so, only one single relative location and/or relative orientation can be allowed or one relative location and/or relative orientation can be selected from multiple possible relative locations and/or relative orientations. For example, thereby at least one or exactly one single degree of freedom of the relative location and/or relative orientation can be variable. Thereby one single or multiple relative locations and/or relative orientations between the light receiving device and the electrode can be defined. For example, the electrode can be arranged in multiple different rotational positions around its longitudinal axis on the apparatus.

In case the electrode is a spatula electrode, for example, in which spatula surfaces are arranged on opposite sides between two side edges, the one or more defined orientations can distinguish from excluded orientations in that the light receiving device is orientated in each of the orientations on one spatula surface respectively or in the only orientation on the spatula surface in an inclined direction, for example. The apparatus can be connectable with a handle and configured to complete the handle on one hand by means of the electrode that is integrated in the apparatus or forms an individual component to form an operable instrument in this manner. The handle can be preferably also completed with an electrode to form an operable instrument, also without the need to attach the light receiving device by means of the apparatus to the instrument. The instrument component, e.g. the handle, itself is accordingly preferably configured to support an electrode.

The apparatus preferably defines a predefined discrete selection, e.g. discrete values and/or ranges separated from one another, and/or—at least in ranges—a continuous selection of possible spatial relative positions of the attached light receiving device relative to the instrument. All relative positions of the selection are characterized in that it is guaranteed that a sufficient amount of light of the light appearance (e.g. a spark or a continuously maintained plasma) created by the instrument can be captured by the light receiving device (if apparatus, instrument and light receiving device are determined for one another). In doing so, it can be ensured that the physician or his assistant attaches the light receiving device always in the correct position on the instrument such that sufficient amount of light is received by the light receiving device in order to guarantee a meaningful light analysis. The selection can include one single possible relative position or multiple possible relative positions. The spatial relative position is defined by the location and orientation of the light receiving device, particularly the light input of the light receiving device, relative to the instrument or the instrument component of the instrument, e.g. the electrode or the handle.

The selection can include a discrete selection of possible relative positions and/or a non-discrete (continuous) selection of possible relative positions. Embodiments are possible in which the apparatus allows only a discrete selection of an endless number of predefined relative positions. Alternatively, embodiments are possible at which the device does not define a discrete subselection, but in which a non-discrete selection or adjustment of relative positions is possible. A non-discrete selection of possible relative positions means particularly that the user can modify the relative position starting from the actual relative position—namely beyond clearance within the actual relative location. In the wake thereof, additional possible relative positions can be passed continuously.

The spatial relative position of the attached light receiving device is defined relative to the instrument by means of the apparatus preferably entirely (relative position of a discrete selection or subselection of one or more relative positions) or, for example, except for one or two degrees of freedom (relative position in a continuous subselection possible relative positions). The apparatus can define the spatial relative position, except for one or two angles, for example. The degree of freedom or one of the degrees of freedom that are not defined or that are defined except for a variety of a value range can be, for example, the angle of light acceptance around the instrument, e.g. around an electrode of the instrument. For example, the apparatus can define the spatial relative position completely (only one single relative location is possible), wherein this can include a slight clearance of the instrument in the or with reference to the apparatus, e.g. in a longitudinal direction of the instrument or the apparatus, a radial direction and/or a rotation direction, which does not affect the light analysis. In each case however, a movability of the light receiving device relative to the instrument, e.g. relative to an electrode of the instrument, is limited by the device in that in all remaining positions of the light receiving device relative to the instrument, e.g. relative to the electrode, a reliable light reception is guaranteed by the light receiving device from the area in which the sparks and/or the plasma is created due to the influence of the instrument. Preferably the light acceptance cone comprises or surrounds the area in which light appearances are created in all of the positions, which the apparatus allows for the light receiving device and the instrument relative to one another.

With embodiments of the inventive apparatus a light receiving device can be used on an instrument for the open surgery, e.g. a radio frequency handle having a radio frequency electrode (also denoted as (RF) applicator), an instrument for the laparoscopy or an instrument for the flexible endoscopy, as for example flexible probes for the argon plasma coagulation.

If the apparatus defines a predefined variety of spatial relative positions of the attached light receiving device relative to the instrument, an erroneous orientation of the light receiving device by the user of the instrument is excluded, who is educated in the medical use of the instrument as physician, but however not in finding a position of the light receiving device relative to the instrument, such that the light receiving device is able to operate. An intervention of the physician by means of an RF surgical instrument can, for example, first mean the opening and exposing the operative site by means of an RF surgical cutting instrument and subsequently the preparation for the tissue distinction. During opening and exposing the operative site, adjustments of electrical parameters for cutting can be partly necessary that allow an efficient hemostasis, but also significant damage. During the tissue distinction, however, only a minor thermal damage is accepted. Also the electrode shape that the surgeon uses for opening and exposing and for tissue distinction can be different. The thermal damage of the tissue (higher energy input) correlates with the contamination of the optical fiber at the distal end. With embodiments of the inventive apparatus the surgeon can attach the light receiving device by means of the apparatus on the handle only if the steps of opening and exposing the operative site are terminated. If for the subsequent tissue distinction an adjustment of the instrument with lower thermal damage is necessary, the danger of contamination of the light input is reduced.

The instrument comprises preferably an electrode that can be applied with RF energy such that the instrument can be used for an RF surgical intervention, independent from the apparatus. The electrode of the instrument, e.g. of the RF applicator, can be exchangeable. The instrument can be an RF applicator having a handle, for example, comprising an electrode. The electrode can be exchangeably attached to the handle. The handle can allow the position and orientation of the electrode except for one single or a discrete variety of possibilities in relation to the handle. As an alternative or in addition, the apparatus can comprise the light receiving device as well as an electrode, for example.

The apparatus is preferably configured to define a spatial relative position of the attached light receiving device relative to the instrument in that the device defines a predefined variety of spatial relative positions of the attached light receiving device relative to the electrode. For this the apparatus can be configured to define a continuous or discrete variety of possible positions of the light receiving device relative to the electrode. The apparatus can be configured to define one single possible spatial relative position for the attached light receiving device relative to the electrode. Due to the definition of a variety of possible relative positions, the orientation and the distance of the light receiving device relative to an area on the electrode shall be defined in which the instrument creates light appearances by means of the electrode. If the orientation and the distance of the light receiving device, particularly the light input of the light receiving device, relative to the electrode is predefined, a specific meaning and quality of the light analysis by means of the light receiving device can be guaranteed.

Preferably the apparatus comprises a mount that limits the movability of the instrument in the mount to a movability in one direction, particularly in axial direction of the instrument, the electrode, the mount and/or the apparatus. Preferably the apparatus defines by means of the mount that the instrument has to be inserted in the direction, particularly axial direction, in the mount during attachment. The mount can be configured to limit movements of the instrument or the instrument component in axial direction toward the distal end of the device.

Preferably the apparatus comprises a unit for subsequent blocking of the axial position of the instrument in the mount, except for at most an axial clearance that does not affect the light analysis, e.g. because the tip of the electrode always remains in a light acceptance angle of the light receiving device despite of the clearance. The unit for subsequent blocking can be configured to limit the movement of the instrument or the instrument component in axial direction away from the distal end of the apparatus.

The unit for subsequent blocking of the axial position preferably comprises a section that is movable laterally relative to the axial direction of the instrument and/or the apparatus and/or the electrode in the course of the attachment of the light receiving device on the instrument. This movable section is preferably configured to engage the instrument by means of a lateral movement of the section after arranging the instrument in the mount in order to block the axial position of the instrument in the mount. The movable section is preferably attached with the remaining apparatus in a flexible manner in order to be able to move the section laterally. This is preferred compared with embodiments in which the movable section is joined or hinged to a further section of the instrument. Preferably the section is resilient such that it automatically moves back in direction of its initial position after the movement of the section against a spring force prior to the arrangement of the instrument in the apparatus. Provided a flexible section, a hinge can be omitted, which simplifies the manufacturing of the device and simplifies sterilizing and/or cleaning of the device.

Preferably the apparatus comprises a flushing channel, wherein the flushing channel is configured to output flushing medium, e.g. carbon dioxide, noble gas, nitrogen or another chemically inert gas or gas mixture, preferably in a laminar manner along the light receiving device passing the light inlet of the light receiving device. In doing so, a contamination of the light inlet of the light receiving device can be avoided, the light inlet of the light receiving device can be cleaned, aerosols between the light inlet and the location of creation of the light can be blown away and/or the instrument, particularly an electrode, can be cooled.

The apparatus is preferably configured to define the spatial position of the flushing channel relative to the light receiving device to a particularly discrete variety of spatial relative positions or one single spatial relative position. The flushing flow is preferably orientated in the direction in which the light receiving device or its light inlet faces, i.e. in the direction of the center axis of the light acceptance angle. In embodiments the flushing flow can be deflected at the light inlet or between the light inlet and the influence area of the RF electrode inclined also up to 90° relative to the optical axis, e.g. by means of a baffle plate or by means of an elevation on one side in the flow channel in order to serve as particle barrier.

In case of a too intensive flow of flushing medium, tissue can be pushed aside thereby. This has to be avoided for high precision, particularly during preparation, i.e. the precise opening or exposure of anatomical structures. On the other hand, the volume flow rate of flushing medium shall be sufficient to reliably avoid contaminations of the light inlet and/or to provide sufficient cooling of the electrode. In addition, gaseous or liquid media, as an example fumes, blood or liquid fat, can be forced away with sufficient flow. If the spatial position of the flushing channel relative to the light receiving device, particularly the light inlet thereof, is defined, it can be operated with precisely adapted volume flow rate of flushing medium that does not result in a precision reducing tissue displacement, but in reliably keeping contaminations away from the light inlet and/or displacement of media affecting capture of light and/or cooling.

Preferably the light inlet of the light receiving device is arranged in the apparatus in order to provide protection of the light inlet from contamination. In doing so, the apparatus is able to shield the light inlet against liquid droplets or particles coming in particular angles without affecting the light incident in the light inlet from other angles. In embodiments having a flushing channel within the apparatus, the light inlet can be, for example, arranged inside the flushing channel or the light receiving device can end inside the flushing channel.

In embodiments having a flushing channel the light receiving device can extend inside of the flushing channel or through the flushing channel, for example. Concurrently the light receiving device is preferably immovably arranged inside the apparatus in one single defined spatial position.

A particularly compact configuration can be obtained, if the light receiving device, e.g. a straight end section of the light receiving device that can adjoin the light inlet—and/or the optical axis of the light receiving device and/or the center axis of the light acceptance angle of the light receiving device and an electrode include an acute angle, e.g. less than 15°.

In embodiments of the apparatus—in case of an established connection between the apparatus and the instrument—a channel section of the fluid channel is preferably configured being limited on one side by a wall of the apparatus and on another side by a first longitudinal side of an electrode and/or an electrode shank of the apparatus. Flushing medium can flow through the channel section. A second longitudinal side of the electrode and/or the electrode shank opposite the first longitudinal side is exposed preferably at least in sections along the channel. While flushing medium flows through the channel section it can cool the electrode at the first longitudinal side. The second longitudinal side is exposed and therefore is in contact with the environment of the apparatus, particularly in contact with air or contact to gaseous or aerosol medium in the environment and can thus dissipate sufficient heat such that the electrode is not excessively heated entirely during operation. The volume flow rate of the flushing medium can thus be adjusted to a rate that guarantees sufficient cooling of the electrode—and preferably also a sufficient shielding and/or cleaning of the light inlet from contamination.

The light receiving device, e.g. an optical fiber, extends preferably from the light inlet continuously inside and/or adjacent to the apparatus—without optical interface therebetween—proximally beyond the apparatus to transmit light for the purpose of analysis. This is advantageous, because an optical interface can contaminate, if for a part of the surgical intervention no light receiving device having a light inlet would be connected thereto.

For example, the instrument can be an RF surgical cutting, coagulation and/or devitalization instrument. For example, the instrument can be configured to operate with gas that can be ionized that is transferred into a plasma condition. Particularly the instrument can be an argon plasma coagulation (APC) instrument. Alternatively or additionally, the instrument can be configured to use a dielectrical barrier discharge.

According to an aspect of the invention, in addition an arrangement of an instrument and an inventive apparatus is provided such that a light receiving device is attached to an instrument in the arrangement by means of the apparatus.

### BRIEF DESCRIPTION OF THE DRAWINGS

Additional features and advantageous embodiments of the apparatus and the arrangement are derived from the following description as well as the figures. The figures show by way of example:

FIG. 1a a perspective illustration of an instrument in the form of an RF applicator that is attached in an embodiment of the inventive apparatus,

FIG. 1b the inventive apparatus according to FIG. 1a in a perspective view in longitudinal section,

FIG. 2a the exemplary inventive apparatus according to FIGS. 1a and 1b in a side view, wherein the distal end section of the apparatus is partly shown in longitudinal section,

FIG. 2b an enlargement of the partly shown longitudinal section of FIG. 2a (part in FIG. 2a illustrated by a circle),

FIG. 3a the embodiment of the inventive apparatus according to FIGS. 1a and 1b in the top view,

FIG. 3b a partly top view on the distal end of the apparatus according to FIGS. 1a and 1b (part illustrated in FIG. 3a by a circle),

FIG. 4a a side view of the embodiment of the instrument according to FIG. 1a in the form of an RF applicator that is attached in the embodiment of the inventive apparatus according to FIG. 1a,

FIG. 4b a top view of the embodiment of the instrument according to FIG. 1a that is attached in the apparatus according to FIG. 1a,

FIG. 4c the instrument in the apparatus according to FIG. 1a in a view in longitudinal section,

FIG. 4d a part of the longitudinal section illustration according to FIG. 4c,

FIG. 4e an alignment structure and a counter-alignment structure,

FIGS. 5a and 5b highly schematical illustrations of possible relative orientations of a light inlet relative to an electrode and

FIG. 6 a further embodiment of an inventive apparatus with a connected example of an instrument for the laparoscopic surgery,

FIG. 7 a further embodiment of an inventive apparatus connected with an example of an instrument for the endoscopic surgery,

FIGS. 8a to 10b a further embodiment of the inventive apparatus having a suction unit,

FIG. 11a a handle part of the instrument,

FIG. 11b an inventive device supporting an electrode,

FIG. 11c a section a part of the longitudinal section illustration according to FIG. 11b,

FIG. 12a an inventive apparatus having an extension element between an electrode and a handle in a perspective view,

FIG. 12b the arrangement according to FIG. 12a in a view in longitudinal section,

FIG. 13 a perspective illustration in part of an area adjoining a distal end of an instrument that is arranged in a further embodiment of an inventive apparatus and

FIG. 14 a longitudinal section through the area of the instrument and the apparatus shown in FIG. 13.

### DETAILED DESCRIPTION

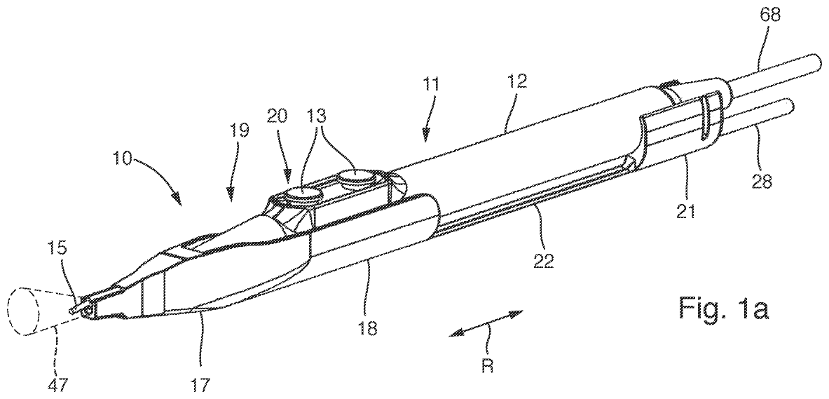

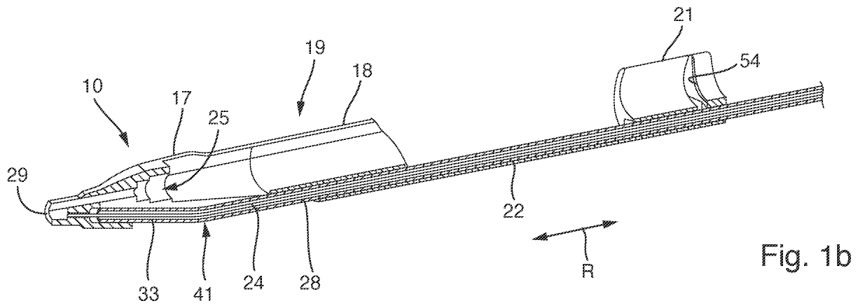

FIG. 1a shows a perspective view of an embodiment of an inventive apparatus 10 in form of a handle part in which an exemplary instrument 11 is held, which is here an applicator for the open surgery. The instrument 11 is preferably an instrument for monopolar RF surgery, in which one pole of the voltage source is connected to a large area neutral electrode (not illustrated) attached to the patient. As an alternative, the instrument 11 can be an instrument for the bipolar RF surgery.

The applicator forming the instrument 11 comprises a handle part 12 having operating elements 13. In the distal end section of the handle part 12 an electrode holding shank 14 having an electrode 15 is mounted. The handle part 12 is connected with an electrical line 68 via which the electrode 15 can be applied with electrical radio frequency power.

The apparatus 10 comprises a distal section 17 in which the applicator 11 is inserted. The proximal end section 18 of the distal section 17 of apparatus 10 comprises a half shell holding the applicator 11. The half shell 18 is open on one longitudinal side 19 (top side) in order to provide access to the operating elements 13 of the instrument 11 at the top side 20 of the instrument 11.

The device further comprises a proximal section 21 that has, according to the example, a half shell form. It is open at the longitudinal side 19 of the apparatus 10 at which the proximal end section 18 is open as well. A proximal section 21 is connected with the distal section 17 of the apparatus 10 by means of a flexible transition section 22. The apparatus 10 surrounds the applicator 11 at least partly in a form-fit manner in order to define the relative position of a light conducting device and/or light receiving device 24 relative to the applicator 11. The light conducting device and/or light receiving device can particularly comprise at least one optical fiber.

The apparatus 10 extends from the proximal end to the distal end substantially in an axial direction R. The distal section 17 and/or the proximal section 21 can have a curved extension around an axis of curvature extending parallel to the longitudinal direction L. According to the example, the transition section 22 is rod-shaped. The transition section 22 is connected with the distal section 17 and/or the proximal section 21 at a central location from which the two shell parts of the distal section 17 or the proximal section 21 curve away in opposite directions in order to form the respective half shell. Particularly the transition section 22 does not form a concave mounting area for the applicator 11, contrary to the distal section 17 and the proximal section 21.

The distal section 17, the transition section 22 and the proximal section 21 consist preferably of the same plastic. The transition section 22 is preferably seamlessly monolithically connected with the distal section 17 and the proximal section 21. The apparatus 10 can be particularly manufactured by means of an injection molding process. The flexibility of the transition section 22 compared with the distal section 17 and the proximal section 21 is achieved according to the example, in that the transition section 22 is formed slimmer than the distal section 17 and the proximal section 21. It can particularly have a small dimension in each direction transverse to its extension ensuring the flexibility. Preferably the apparatus 10 or at least the distal section 17 is transparent in order to provide an improved view onto the operative site.

As particularly illustrated in FIGS. 1b, 2b and 4d, the distal section 17 of the apparatus 10 provides a channel-like first mount 25 for a form-fit section 26 of the applicator 11. The first mount 25 (that can also be denoted as first mounting channel) is preferably closed along the periphery (in all radial directions). The first mount 25 defines by means of form-fit an orientation of the electrode 15 of the instrument 11 in the apparatus 10 along a longitudinal axis L of the first mount 25.

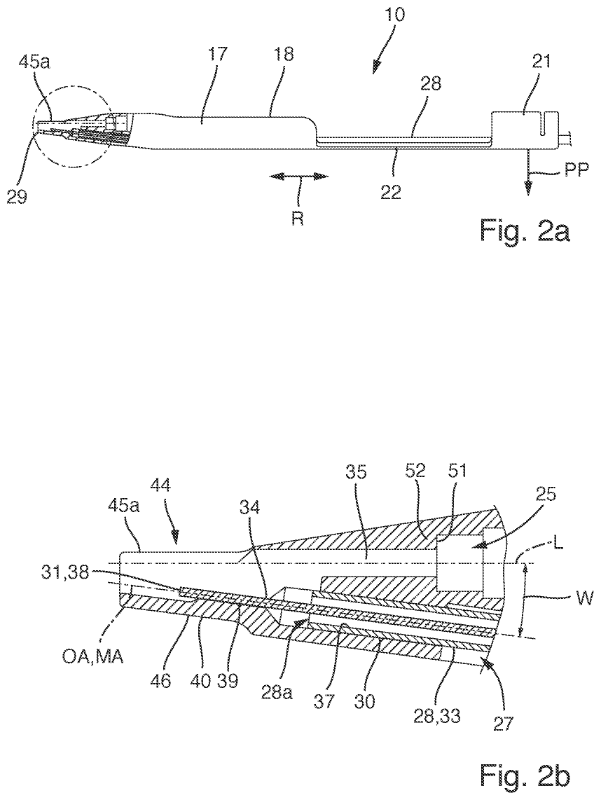

The apparatus 10 comprises in addition, as particularly also shown in FIG. 2b by way of example, a second mount 27 configured in a channel-like manner and provided for a line 28, e.g. a hose or a tube, through which the at least one optical fiber—i.e. one single optical fiber or a bundle of optical fiber strands—extends in direction toward the distal end 29 of the distal section 17 of the apparatus 10 forming a light receiving device 24. The line 28 is attached in the second mount 27 preferably by force-fit and/or friction-fit with the wall surface 30 of the second mount 27 against movements of the line 28 inside the second mount 27. The line 28 forms a conduction channel (flushing channel) and serves to conduct a flushing medium for cleaning and/or shielding a light inlet 31 of the optical fiber 24. The optical fiber 24 serves to capture light of light appearances that are created by the electrode 15 of the applicator 11. The location of the optical fiber 24 in the apparatus 10 is preferably completely defined.

The line 28 extends in a protected manner laterally next to the applicator 11 between the flexible transition section 22 and the applicator 11 through the half shell-shaped proximal section 21 of the distal section 17 of the apparatus 10.

The second mount 27 for the line 28 is configured to guide it in sections substantially parallel to the applicator 11 in direction toward the distal end 29 of the apparatus 10. As particularly illustrated in FIGS. 1b and 2b, the second mount 27 is further configured to guide a line section 33 of line 28 adjoining the distal end 32 of line 28 in an acute angle to the longitudinal axis L of the first mount 25. As particularly illustrated in FIG. 2b, the end section 34 of the optical fiber 24 adjoining the light inlet 31 of the optical fiber 24 or the optical axis OA thereof and/or the center axis MA of the light acceptance angle 47 of the optical fiber 24 also includes an acute angle W with the longitudinal axis L of the mounting bore 35 of the first mount 25 for the electrode 15 or the electrode holding shank 14—and thus also with the longitudinal axis ELA of electrode 15 that coincides with the longitudinal axis L of first mount 25. The acute angle in which the line section 33 is guided toward the longitudinal axis L of the first mount 25 corresponds preferably with the angle W between the center axis MA of the light acceptance angle 47 and the longitudinal axis ELA of electrode 15.

The optical fiber 24 can be centrally held in the line 28 or can substantially coaxially follow its extension. For example, holding elements (not shown) can serve for this purpose that are arranged inside the line 28 that allow a flushing medium flow through line 28 to the distal end 29 of the apparatus 10 and concurrently keep the optical fiber 28 at a distance to the inner wall surface 37 of line 28. As an alternative, the fiber 24 can be placed loosely in the line hose 28, for example, wherein an end section 34 of the fiber can be rigidly connected with the apparatus 10, as described below. For example, the optical fiber 24 is preferably secured also against twist of the optical fiber 24 around a longitudinal axis of the optical fiber 24. The volume flow of flushing medium is guided in the embodiment substantially concentrically relative to the optical fiber 24. As an alternative, the volume flow can be laterally supplied at the distal end 38 of the optical fiber 24. In both embodiments the flushing flow flowing around the distal end 38 of the optical fiber 24 serves to avoid contamination by spraying tissue liquid droplets or flying particles on the light inlet 31 of the optical fiber 24.

As illustrated in FIGS. 2a and 2b, the optical fiber 24 projects from the distal end 32 of line 28 such that an end section 34 of the optical fiber 24 extends in the acute angle W2 relative to the electrode holding shank 14 adjacent thereto. The optical axis OA (e.g. longitudinal axis of the optical fiber) and/or the center axis MA of the light acceptance angle 47 thus include the acute angle W2 with the electrode longitudinal axis ELA of, for example, less than 15°, illustrated 7.5°. The end section 34 of the optical fiber 24 that projects from the distal end 32 of line 28 can be placed for this purpose on a support section 39 of mounting wall 40 of the distal section 17 of the apparatus 10 appearing base-like or projection-like in the longitudinal section through the device (FIG. 2b) and can be rigidly connected therewith, e.g. in a substance bond, in order to stabilize the end section 34 of the optical fiber 24 in this position. The optical fiber 24 is pressed by means of its inherent tension due to the bend 41 of the optical fiber 24 in the line 28 against the support section 39. Thereby the position of the optical fiber 24 relative to the longitudinal axis L of first mount 25 is defined. This also applies for the direction orthogonal to the drawing plane in FIG. 2b. The mounting area 42 of the support section 39 can be, for example, half shell formed in order to define the position of the optical fiber 24 together with the inherent tension of the optical fiber 24 transverse to the plane that is spanned by the end section 34 of the optical fiber 24 and the longitudinal axis L (axial direction) of first mount 25 or the axial direction of electrode 15.

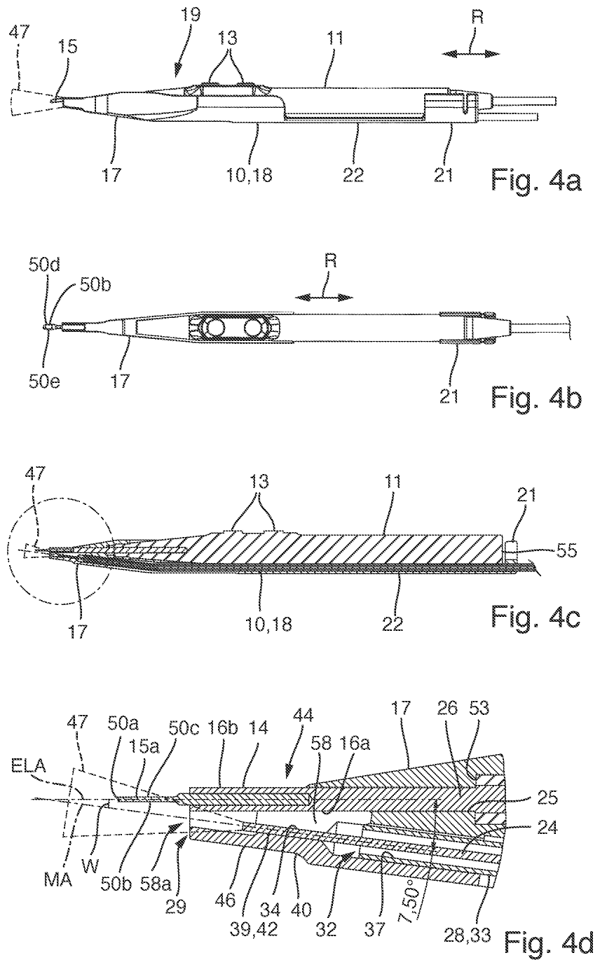

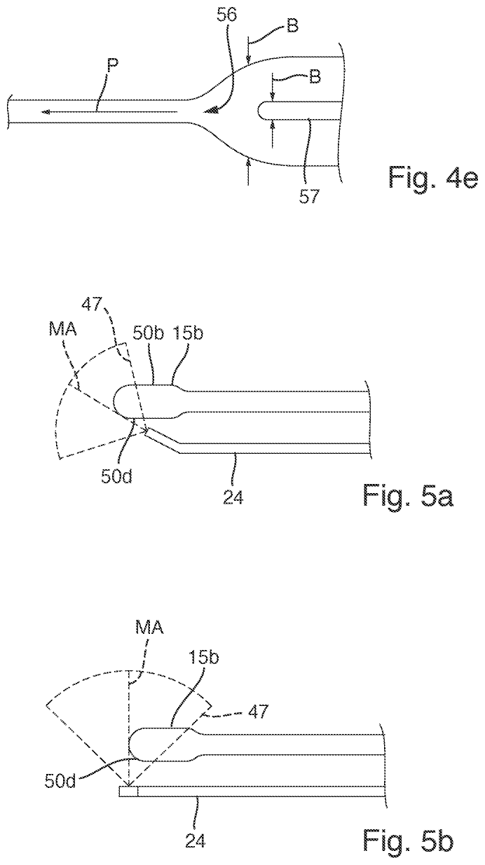

In order to counteract contamination of the optical fiber 24, the optical fiber 24 preferably does not end flush with the apparatus 10 and also does not protrude from the apparatus 10. Rather the optical fiber 24 ends preferably relative to the distal end 29 of apparatus 10 offset backwardly inside a channel-shaped region 44, as illustrated in FIGS. 2b, 4d, in order to avoid a direct contamination of the optical fiber 24 from a specific angle.

The region 44 of the apparatus 10 is preferably open to one side. For example, if also the proximal end section 18 of the distal section 17 is open, the region 44 can be open to the same side (e.g. toward “the top”, for example in FIGS. 1b and 2a). This is particularly apparent from FIGS. 1b, 2a and 2b in which the apparatus 10 is illustrated without a connection established between the apparatus 10 and the instrument 11. Toward the opposite side (“downward”) the region 44 is preferably closed on the contrary. The half shell formed by the proximal end section 18 of the distal section 17 can be configured without through openings and particularly laterally closed in embodiments.

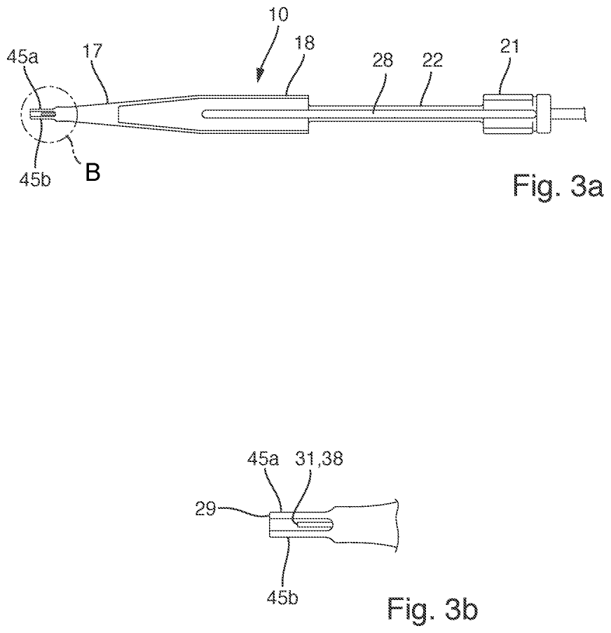

The apparatus 10 comprises two side wall sections 45a, 45b in the region 44 that extend laterally past the optical fiber 24 such that the distal end 38 of the optical fiber 24 is offset backwardly relative to the distal end 29 of apparatus 10. In doing so, the light inlet 31 is protected at the face of the optical fiber 24 at least from particles and/or droplets that move from specific angles in direction toward the light inlet 31. The side wall sections 45a, 45b are connected via a bottom wall section 46 such that the region 44 or the apparatus 10 can have a U- or V-shaped cross-section there, for example, as apparent from FIG. 2b in connection with FIG. 3a. The support section 39 is at least partly formed by the bottom section 46. The free end 38 of the optical fiber 24 can slightly project beyond the support section 39 and thus be arranged slightly distant from the side wall sections 45a, 45b, 46 of apparatus 10 in all radial directions inside region 44. This avoids a too large shading of the light acceptance angle 47 of the optical fiber 24 by the apparatus 10.

The light acceptance angle 47 is drawn in dashed lines in FIGS. 1a, 4c as well as 4d for illustration purposes. The light acceptance angle 47 comprises all directions from which the optical fiber 24 can capture light in the light inlet 31 and can transmit it to an analysis device by means of which the light can be analyzed. In the electrode holding shank 14 a working section 15a of electrode 15 is fixed. As illustrated, the light acceptance angle 47 comprises the tip or the distal end 50a of electrode 15 or the working section 15a. The position and/or orientation of electrode 15, particularly its working section 15a or tip 50, is unambiguously defined relative to the form-fit section 26 of instrument 11. In addition, the position and/or orientation of the optical fiber 24 relative to the first mount 25 is unambiguously defined. The form-fit section 26 provides together with the first mount 25 for a defined position of the optical fiber 24 relative to the electrode 15, particularly to its working section 15a, if a connection between apparatus 10 and instrument 11 is established. By means of the form-fit between apparatus 10 and instrument 11 it is ensured that the location and/or orientation of the optical fiber 24 allows a light analysis of light appearances—that are created by means of the working section 15a of electrode 15—if the connection between apparatus 10 and instrument 11 is established. In embodiments the location of the electrode 15 relative to the instrument 11 and/or the apparatus 10 can be defined, however, the orientation of the electrode 15 around its longitudinal axis ELA can be freely selectable within a continuous variety.

For example, a section of the electrode 15 can be part of the form-fit section 26 of the applicator 11. A section of the electrode holding shank 14 of electrode 15 of the applicator 11 is part of the form-fit section 26 in the illustrated embodiment. The form-fit section 26 comprises a shape that fits a respective complimentary counter-shape of the first mount 25 of apparatus 10. In the illustrated embodiment the electrode holding shank 14 has a cylindrical shape that fits in the cylindrical form (bore) of first mount 25. Alternatively to the cylindrical form of form-fit section 26 and the first mount 25, they can have, e.g. polyhedral shapes fitting one another, e.g. a rectangular shape. By means of the pair of form-fit section 26 and first mount 25 the relative movability of the form-fit section 26 inside the first mount 25 and relative to the first mount 25 is blocked in all directions orthogonal to the center axis MA of the first mount 25 or to the longitudinal axis ELA of the electrode holding shank 14 and thus also relative to the working section 15a of electrode 15. By means of the first mounting channel 25, that can be closed in all radial directions, the position of electrode 15 relative to the end section 34 of the optical fiber 24 is defined radially in all directions.

A respective first stop 51 (see particularly FIG. 2b) is formed by means of a wall section 52 of apparatus 10. It is arranged axially opposite a first counter stop 53 (see particularly FIG. 4d) of applicator 11, if applicator 11 is moved forward in the first mount 25. The first counter stop 53 can be formed, for example, by the electrode holding shank 14 or, as illustrated, by an electrode shank overmold that is a section of the handle part 12. The first stop 51 and the first counter stop 53 serve to define the position of the optical fiber 24 in direction toward the electrode tip 50a or in axial direction of electrode 15 or the electrode holding shank 14. By means of the first stop 51 and the first counter stop 53, the maximum distance between the electrode tip 50a and the light inlet 31 is defined. The position of the optical fiber 24 relative to the electrode 15 is preferably defined such that the optical fiber 24 faces an area in front of the tip 50a of electrode 15. The optical axis OA of the optical fiber 24 intersects an area in front of the electrode tip 50a—preferably the longitudinal axis ELA of electrode 15—in a point in front of tip 50a of electrode 15.

A second stop 54 for axial securing and particularly positioning of applicator 11 is formed on the proximal section 18 of apparatus 10. A second counter stop 55 of instrument 11 is formed by the proximal end of applicator 11 in the embodiment (see FIG. 4c). The second stop 54 and the second counter stop 55 avoid an undesired movement of applicator 11 away from the distal end 29 of apparatus 10. In doing so, a minimum distance between the electrode tip 50a and the light inlet 31 of optical fiber 24 can be maintained. The position of the light inlet 31 of the optical fiber 24 relative to the tip 50a of electrode 15 is defined by means of the stop/counter stop pairs 51, 53, 54, 55 axially except for at most a clearance between the instrument 11 and the apparatus 10 in axial direction R that does not affect the light analysis by means of the optical fiber 24. Particularly the electrode tip 50a is kept inside the light acceptance angle 47 of optical fiber 24, as illustrated in FIGS. 4c and 4d, if the connection between the apparatus 10 and the instrument 11 is established. The light acceptance angle 47 of optical fiber 24 is preferably orientated such that the tip 50a of RF electrode 15 is located in the center of the light acceptance angle 47 to capture light appearances on the left and on the right (on one narrow side as well as on the opposite narrow side) around the electrode 15.

In order to capture as much light as possible from the RF spark—that forms a divergent light source—the diameter of the light inlet 31 of the optical fiber 24 should be as large as possible and the light inlet 31 should be positioned as close as possible to the location of creation of the light appearance, e.g. an RF spark, at the electrode tip 50a in a manner maintaining the position. In order to counteract a contamination of optical fiber 24, e.g. by fume particles, aerosols or by spraying tissue particles, e.g. fat droplets, the light inlet 31 of optical fiber 24 would have to be located, however, as far as possible away from the location of creation of the light appearance. By means of defining the position of the applicator 11 between first stop 51 and second stop 54 and relative to the apparatus 10, an intensive light reception on one hand and an at most low contamination on the other hand is guaranteed.

The working section 15a of electrode 15 can be spatula-shaped (as illustrated) or needle-shaped, for example. The electrode 15 projects from the distal end 29 of apparatus 10 or protrudes beyond the distal end 29 of apparatus 10 in distal direction. For example the electrode 15 is orientated such that a flat side 50b (that can also be denoted as spatula side) of electrode 15 obliquely faces the light input 31 of optical fiber 24. Alternatively, the electrode 15 can be orientated such that the light input 31 faces a narrow side 50d, 50e (that can also be denoted as edge) of electrode 15. The rotational orientation of apparatus 10 or optical fiber 24 around the longitudinal axis ELA of electrode 15, e.g. the longitudinal axis of the spatula-formed working section 15a, can be defined by means of an alignment structure 56 and a counter alignment structure 57, as an example, e.g. during connection of the instrument 11 with the apparatus 10.

Exemplary alignment structure 56 and counter alignment structure 57 are shown in FIG. 4e. A pair of one alignment structure 56 and one counter alignment structure 57 can consist, e.g. of a cavity 56 and a projection 57, wherein the projection 57 has to be moved relative to the cavity 56 into the cavity 56. As illustrated, the cavity 56 and/or projection 57 can be configured such that their width B decreases in movement direction from the inlet of cavity 56. An alignment structure 56 at the periphery of applicator 11 engages the counter alignment structure 57 of apparatus 10, e.g. during forward movement of electrode 15 in the first mount 25, such that the orientation of the optical fiber 24 around the longitudinal axis ELA of electrode 15 is defined during connection of instrument 11 with apparatus 10. Obviously the alignment structure 56 can be alternatively formed on the apparatus 10 and the counter alignment structure 57 can be alternatively formed on the instrument 11. Arrow P illustrates the movement direction in which the instrument 11 is moved forward in the apparatus 10 during establishing the connection.

During assembly of the apparatus 10 and instrument 11 during operation, the user of the device, e.g. a surgeon or an assistant of the surgeon, can proceed for example as follows: The user presses the proximal section 21 of apparatus 10 laterally away from a longitudinal axis of the distal section 17 or the longitudinal axis L of the first mount 25 against a spring force of transition section 22 (see arrow PP in FIG. 2a), such that the user can now move the electrode 15 along the longitudinal axis L in the first mount 25. Because the first mount 25 holds the electrode holding shank 14 by precision fit, it is not possible to align the applicator 11 relative to the apparatus 10 such that the electrode 15 can be moved in the first mount 25 without laterally pushing away the proximal section 21. No later than when the first counter stop 53 and the first stop 51 abut against one another, the user can lock the applicator 11 in the apparatus 10 axially between the first counter stop 53 and the second counter stop 55 by releasing the counter movement of the proximal section 21 that is carried out by the proximal section 21 automatically due to the spring force. Thereby the transition section 22 releases and moves the proximal section 21 laterally in direction toward longitudinal axis L. In addition or as an alternative to an attachment of apparatus 10 to one another by locating the instrument 11 in the apparatus 10 it is also possible, e.g. to attach the apparatus 10 with one or more clamps or clips having elastically flexible sections to the instrument 11 (not illustrated), wherein the elastically deformable sections clamp the apparatus 10 and/or instrument 11 there between.

If the instrument 11 is arranged in the apparatus 10, the electrode 15 or electrode holding shank 14 largely closes the region 44 of apparatus 10 in which the optical fiber 24 ends relative to the bottom wall section 46 as by way of example illustrated in FIGS. 4b, 4c and 4d. The electrode 15 and the apparatus 10 thus limit a channel section 58 between the outlet of line 28 at its distal end 32 and the distal end 29 of apparatus 10. During operation of instrument 11 with apparatus 10 the channel section 58 can be supplied with flushing medium via line 28. The flow or flushing medium touches the bottom side 16a of electrode holding shank 14 and/or electrode 15 limiting the channel section 58 and cools it. The top side 16b of electrode holding shank 14 is preferably exposed on the other side adjacent the channel section 58 so that electrode 15 dissipates sufficient heat. Between the top side 16b and the environment of apparatus 10 or instrument 11, particularly no wall section of apparatus 10 is arranged.

The surgeon can operate with instrument 11 or apparatus 10, e.g. as follows: The applicator is preferably usable also without the apparatus 10. During surgical use of applicator 11 the user guides the applicator 11by holding it with handle part 12 and influences thereby the tissue by means of the applicator 11. For example, the surgeon can open and expose the operative site in an RF surgical manner by means of the applicator 11 without apparatus 10 being connected therewith. In this phase of the medical intervention no light analysis is usually required. An unnecessary contamination of the optical fiber 24 can thus be completely avoided in this phase. If the surgeon has prepared the location at which the preparation intervention shall be executed in the described manner, he/she or a surgery assistant can connect the apparatus 10 and thus the optical fiber 24 quickly and in the correct position with the instrument 11. A reliable light analysis is now possible by means of the optical fiber 24.

A system comprising the apparatus 10 can be configured such that the flow or flushing medium is automatically switched on prior to supply of applicator 11 with electrical radio frequency (RF) energy, if the apparatus 10 is connected with instrument 11, in order to be able to counteract a contamination from the outset by a flow around the optical fiber 24 at the distal end. For example, a gas can be used as flushing medium. Alternatively, the system can be configured, for example, such that—provided that instrument 11 is connected with apparatus 10—the supply of the electrode 15 with RF energy is only released, if line 28 is supplied with flushing medium. The flushing medium flow slows down droplets and/or flying particles sprayed away from the location of intervention and deflects them as far as possible, such that they do not hit the light inlet 31 of optical fiber 24. The mass or volume flow of flushing medium can be preferably adjusted to a value or limited to a maximum value by the system, at which the target tissue or the target structure on which the intervention shall be carried out is displaced in a manner that does not affect the surgical precision, but reliably slowing down and/or deflecting particles and/or droplets. Also a gas embolism is preferably avoided by means of the adjustment or limitation of the maximum volume flow and pressure by means of the system. In narrow body cavities the volume flow of flushing medium can in addition displace fume and/or liquid media such as blood or molten fat and thus allow an improved view on the operative site.

The surgeon can grab the assembled group of instrument 11 and apparatus 10 comprising the handle 12 of instrument 11 at the apparatus 10 for operation. Now the apparatus 10 serves at least as a part of a handle piece for handling the assembled group. Now the RF energy supply of electrode 15 is switched on again and the surgeon can continue working on the operative site with the instrument. Light created thereby, e.g. spark light, enters into the light inlet 31 and is analyzed by means of a not illustrated analysis device in order to provide information about the treated tissue to the surgeon, for example.

While FIGS. 1a to 4e shown an embodiment of the apparatus 10 in which the light receiving device 24 faces obliquely onto the flat side 50b of the working section 15a of electrode 15 that is the flat side 50b is obliquely facing the light inlet 31, the light inlet 31 of the light receiving device 24 can also face a narrow side (or edge) 50d of a spatula-shaped working section 15a of an electrode 15. Such a relative position of working section 15a of electrode 15 and light receiving device 24 is shown in FIG. 5a based on an example. The center axis MA of the light acceptance angle 47 is orientated in an acute angle relative to the longitudinal axis ELA of electrode 15.

FIG. 5b shows another example in which the light receiving device 24 is arranged in such a position relative to the electrode 15 in that the center axis MA of the light acceptance angle 47 extends orthogonal to the longitudinal axis ELA of electrode 15.

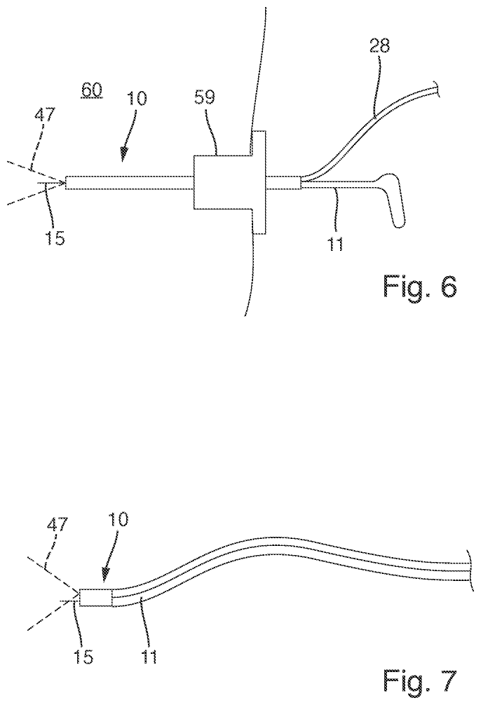

FIG. 6 shows an embodiment of an apparatus 10 for attachment of the light receiving device 24 defining a light acceptance angle 47 for light analysis on an instrument 11 for the laparoscopy that is inserted through a trocar 59 in the abdominal cavity 60. The instrument 11 comprises an electrode 15, which can be supplied with electrical RF energy. The trocar 59 provides access for the instrument 11 with attached apparatus 10, wherein the apparatus 10 and the instrument 11 are configured that the access around the apparatus 10 and instrument 11 being introduced remains sealed such that no gas can escape in an undesired manner from the abdominal cavity 60 through the trocar 59. The geometric outer contour is preferably round, so that the access through the trocar 59 is respectively sealed. The instrument 11 and the optical fiber 24 can be guided in a parallel manner inside a tube, e.g. a liner tube, in order to avoid a mechanical damage. For example, the apparatus 10 can be an adapter that can be attached by a user of the instrument 11 on the instrument 11 and can be preferably released from the instrument 11 or can be formed on the instrument 11 itself. For example, the apparatus 10 is an opening or channel on the instrument 11 in which an optical fiber 24 can be inserted or clipped in, if necessary.

FIG. 7 shows an embodiment of an apparatus 10 for attachment of a light receiving device 24 for light analysis on an instrument 11 for the endoscopic surgery. For example, the instrument 11 can be a flexible probe having an electrode 15 that allows cutting and/or coagulating by means of an electrode 15 supplied with RF energy, the light appearances thereof can be examined by the light receiving device 24. The instrument 11 can be an argon plasma coagulation probe, for example. Alternatively or additionally, the instrument 11 can be configured to use a dielectrical barrier discharge.

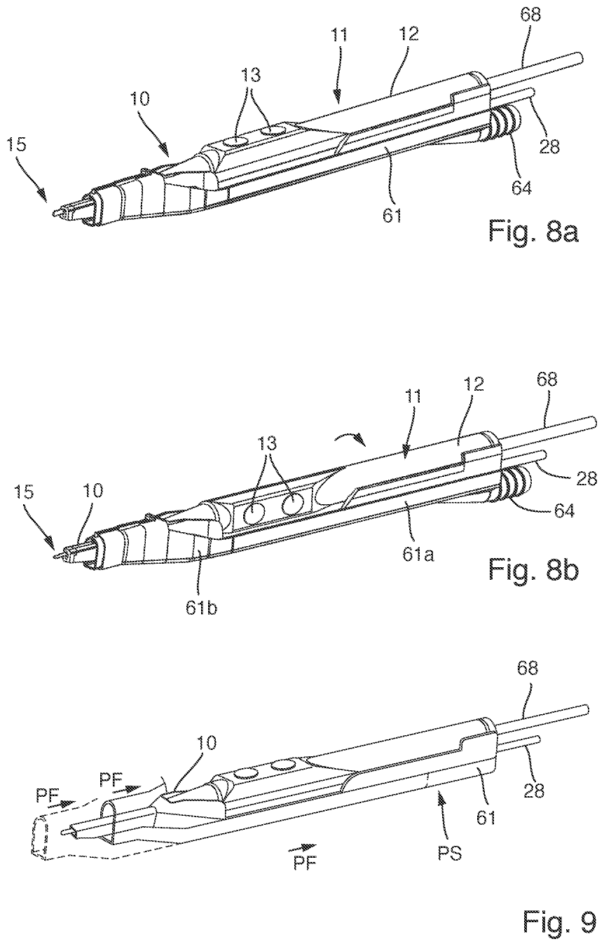

FIGS. 8a to 10b show an example of an embodiment of apparatus 10 that is configured to be connected with a suction unit 61 as an option. The suction unit 61 can be preferably connected with the apparatus 10 during surgery by the surgeon or his assistant. As shown in FIG. 9 by arrows PF, the suction unit 61 is preferably slideably moveable on the apparatus 10, e.g. if the apparatus 10 is connected with the instrument 11. The suction unit 61 is preferably connectable with the apparatus 10 without use of tools and can be non-destructively disconnected from the apparatus 10. As indicated by arrow PS, the suction unit 61 can have a proximal section 61a that is flexibly elastically connected with distal section 61b and that has to be first bent away for the connection of the suction unit with the device and after slideably moving suction unit 61 and apparatus 10 into one another and elastical release in the initial position attaches the apparatus 10 together with the distal section 61b in a form-fit manner in the suction unit 61. This corresponds to the preferred attachment of apparatus 10 on handle 12, as described above. It is alternatively or additionally also possible, for example, to attach the suction unit 61 with one or more clamps or clips having elastically flexible sections on the apparatus 10 and/or the instrument 11 (not shown), wherein the elastically deformed sections clamp the apparatus 10 and/or the instrument 11 therebetween.

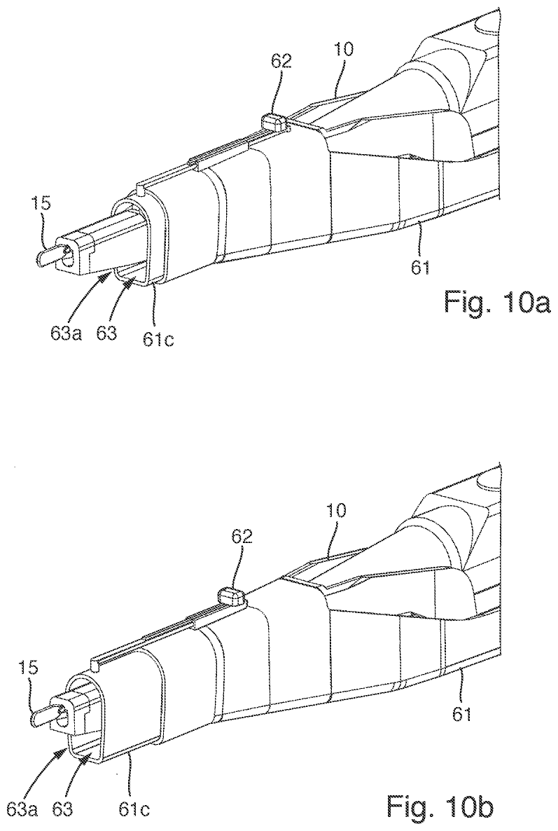

Preferably the suction unit 61 comprises an extension section 61c that is guided in the distal section 61b in a telescopic manner and is displaceable by a forward slideable movement of a slide section 62, if required, in distal direction from a proximal position (shown in FIG. 10a) in a distal position (shown in FIG. 10b).

The suction unit 61 can be transparent at the distal end in order to provide an improved view on the operative site. For example, the suction unit 61 consists or, if provided, at least the extension section 61c of transparent plastic. In prefer embodiments the suction unit 61 as well as the apparatus 10 are transparent at their distal ends.

As an alternative to the embodiment described above, the suction unit 61 can also be an undetachable part of apparatus 10.

The suction unit 61 serves to distract fume from the operative site created during RF surgical application by suction. The suction unit 61 comprises suction channel 63 that extends in the illustrated embodiment through the distal section 61b and the extension section 61c up to a suction opening 63a at the distal end of the suction unit 61. The apparatus 10 limiting the flushing channel 58 and containing the optical fiber 24 extends through the suction unit 61 and in the illustrated embodiment through the suction channel 63 and particularly through the suction opening 63a. While the electrical line 68 for supply of RF power (RF supply line), the line for the flushing medium and the suction line 64 are illustrated in FIGS. 8a to 8b next to one another extending toward the handle 12, the apparatus 10 or the suction unit 61, the line 28 for the flushing medium that preferably comprises also the optical fiber 24, can be proximal from the handle 12 arranged inside the suction line 64, for example.

In this embodiment the suction opening 63a is compared to mouth of flushing channel 58a offset in proximal direction in each position of the extension section 61c. In FIG. 4d the mouth of flushing channel 58a is, for example, the mouth of channel section 58 at the distal end of the apparatus 10. Preferably the opening area of mouth 58a of flushing channel 58 at the distal end 29 of apparatus 10 and/or the opening area of opening 28a of line 28 for the flushing medium is at the distal end 32 thereof smaller than the free opening area of suction opening 63a of suction channel. In this manner a relatively high flow velocity can be achieved in the mouth 58a of flushing channel 58 and/or the opening 28a of line 28. However, the system for avoiding inflation or expansion of tissue can be configured or adjusted, such that the suction volume flow rate (e.g. 50-120l/min) into the suction channel can be, for example, at least ten times higher than the flushing volume flow rate (e.g. about 1 l/min) out of the flushing channel 58. This is particularly advantageous, if the system is configured or adjusted that concurrently flushing with flushing medium and distraction by suction by means of the suction unit 61 is provided. The free opening area of the suction opening 63a of suction channel 63 surrounds electrode 15 and the part of the apparatus 10 at the distal end 29 in which the flushing channel 58 and the light receiving device 24 are arranged that extend through the suction opening 63a of suction channel 63.

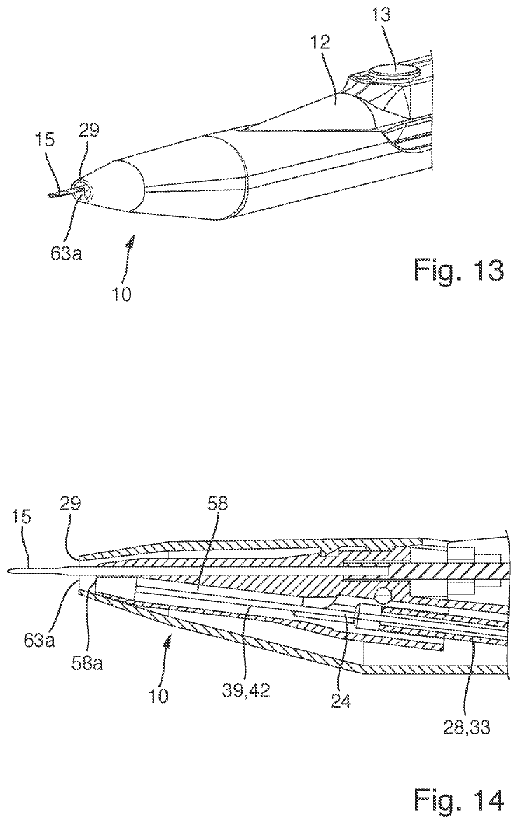

In another embodiment the suction opening 63a can be arranged closer to the distal end of electrode 15 than the mouth 58a of flushing channel 58. Such an embodiment is illustrated in FIGS. 13 and 14. In this embodiment other volume flows for the flushing medium or the suction can be used. For example, the volume flow of the suction can be 6-8 times larger than the volume flow of flushing medium or flushing gas. In one embodiment the suction volume flow rate can be 50 l/min and the flushing volume flow rate of flushing medium can be about 7 l/min.

All indications in liter for the volume flows refer to the standard volume (standard liter) of the fluid.

Independent from whether apparatus 10 is connectable with a suction unit 61 or whether the suction unit 61 is an undetachable from apparatus 10, the apparatus 10 can in some or all embodiments define a discrete variety of possible positions of the light receiving device 24 relative to the applicator 11. If the applicator 11 defines a discrete variety of possible positions of electrode 15 relative to the handle 12, the apparatus 10 provides by means of handle 12 a discrete number of possible positions of the light receiving device 24 relative to the electrode 15. All positions of the variety are characterized in that it is guaranteed (if apparatus 10, instrument 11 and light receiving device 24 are determined for one another) that a sufficient amount of light of the light appearance created by instrument 11 can be received by the light receiving device 24. The position (0° location) shown in FIG. 8a distinguishes from the position (90° location) according to FIG. 8b by a different rotational orientation (rotated about 90°) of apparatus 10 and thus the light receiving device 24 around the longitudinal axis ELA of electrode 15. For example, the discrete positions can be defined by form-fit in rotational direction around the longitudinal axis ELA of electrode 15 by means of structures 56, 57, as illustrated in FIG. 4e. The instrument 11 shown in FIGS. 8a and 8b is preferably configured like the instrument 11 according to FIGS. 4a to 4d. Only the orientation of electrode 15 is such that in the 0° position the light receiving device 24 faces on a narrow side 50d of electrode 15. In the 90° position the light receiving device 24 faces accordingly on the flat side 50b of the spatula-formed electrode 15. The surgeon can thus select during surgery whether the light receiving device 24 shall face directly on an edge 50d, 50e of spatula-formed electrode 15 (FIG. 8a) or whether the light receiving device 24 shall be able to approximately equally receive light from both edges 50d, 50e (FIG. 8b). The latter can be advantageous, if the surgeon wants to use both edges 50d, 50e for operation. The former can be advantageous, if the surgeon wants to use only the edge 50d for operation, on which the light receiving device 24 is orientated, because then it can receive more light from edge 50d as from the opposite edge 50c.

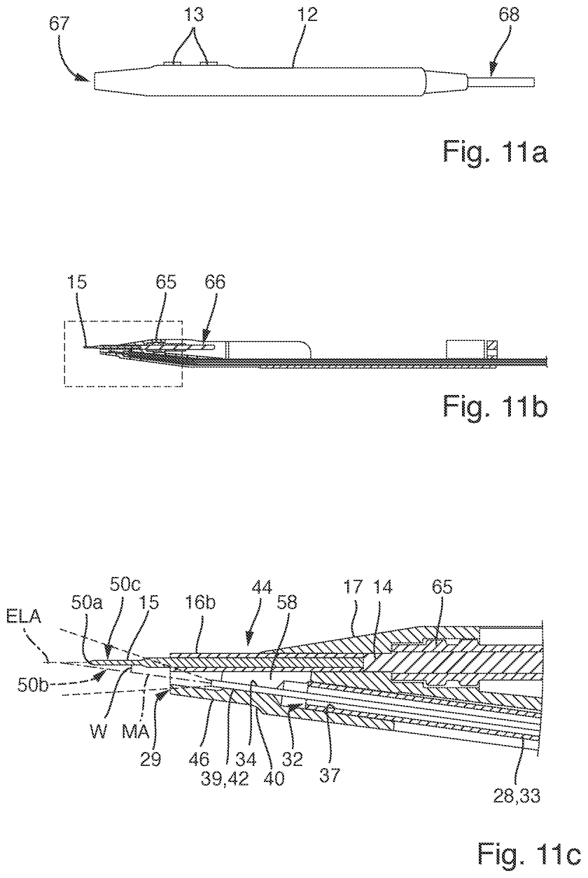

For the embodiment illustrated in FIGS. 11a to 11c of apparatus 10 reference can be made to the explanations with regard to the embodiment according to FIGS. 4a to 4d, wherein the embodiments distinguish from each other in that the apparatus 10 according to FIGS. 11a to 11c comprises an electrode 15. The electrode 15 is supported by apparatus 10 (compare longitudinal section views in FIGS. 11b and 11c) without the requirement that the apparatus 10 has to be connected with handle 12. The apparatus 10 defines particularly the position of the light receiving device 24 relative to the electrode 15, except for such in which the light receiving device 24 faces on a flat side 50b or 50d of electrode 15—or as an alternative except for such in which the light receiving device 24 faces on an edge 50d or 50e of electrode 15. Particularly the apparatus 10 can define one single possible position of electrode 15 relative to the light receiving device 24. The working section 15a of electrode 15 is held in an electrode holding shank 14 that is held in a mounting bore 35. By means of a coupling element 65, the position of the electrode holding shank 14 and thus the electrode 15 relative to the apparatus 10 and thus relative to the optical fiber 24 can be unchangeably provided. The electrode holding shank 14 that can be a one-piece or a multiple-piece element, can comprise a form-fit structure 66 for mechanical and electrical coupling of handle 12 with electrode holding shank 14.

The respective handle 12 is illustrated in FIG. 11a in side view as separate instrument component. The assembly of apparatus 10 and handle 12 can be carried out during surgery, as described above, with the difference that an operable instrument 11 of handle 12 and electrode 15 is created during assembly. Thereby the electrode holding shank 14, in which the electrode 15 is held, is inserted in the mount 67 of handle 12 and for example immovably attached by means of form-fit structure 66. Thereby the electrode holding shank 14 is preferably electrically connected with the RF supply lines 68 to the handle 12. The electrode 15 can then be supplied with electrical energy via electrode holding shank 14. Embodiments of the example according to FIGS. 11a to 11c can allow that handle 12 and apparatus 10 can be combined according to FIGS. 8a and 8b, e.g. in rotationally offset positions, e.g. about 90°, without changing the relative position of the light receiving device 24 and the electrode 15 (e.g. the light receiving device 24 faces the flat side 50b of electrode 15).

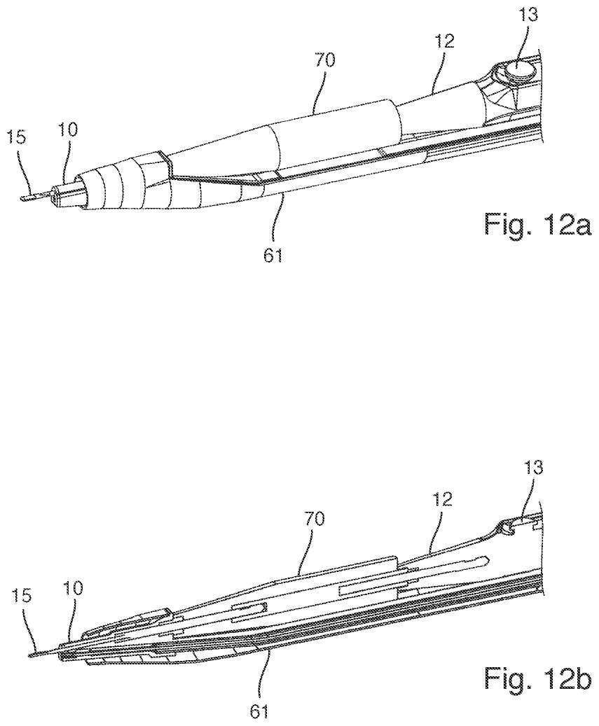

FIGS. 12a to 12b show a handle 12 coupled with an inventive apparatus 10. Between the handle 12 and the distal end of apparatus 10 an extension element 70 is arranged that is held in the apparatus 10. The extension element 70 couples the handle 12 with the apparatus 10 mechanically and the RF supply line 68 electrically via a line part 71 with the electrode 15. The electrode 15 can be supported by extension element 70 or apparatus 10. The above explanations with reference to embodiments in which the electrode 15 is supported by an instrument component of instrument 11, for example the handle 12, can be referred to for examples of embodiments in which the electrode 15 is supported by extension element 71. The above explanations with regard to embodiments in which the electrode 15 is supported by apparatus 10 can be referenced for examples of embodiments in which the electrode 15 is supported by apparatus 10.

An inventive apparatus 10 is provided for attachment by a user of a surgical instrument 11 of a light receiving device 24 for a light analysis on the instrument 11 or an instrument component 12 of instrument 11by the user of the surgical instrument 11. The apparatus 10 is preferably configured to releasably attach the light receiving device 24 on the instrument 11 or the instrument component 12. Preferably the apparatus 10 is configured such that the apparatus 10 can be repeatedly released and can be repeatedly used. In simple cases the instrument can be a handle 12 having an electrode 15. Embodiments of the apparatus 10 comprise a light receiving device 24 as well as an electrode 15 attached relatively thereto, wherein the apparatus 10 can be attached on the handle 12. Embodiments are possible in which the electrode 15 is not supported by apparatus 10, but by the instrument 11. However, embodiments are preferred in which the electrode 15 is a non-destructively releasable part of the apparatus 10 or is immovably installed in the apparatus 10. Embodiments are preferred in which the apparatus 10 is attachable (and preferably releasable again) by the surgical user or his assistant on the instrument 11 or the instrument component 12 and forms an adapter for attachment of a light receiving device 24 to the instrument 11 or the instrument component 12. In other embodiments the apparatus 10 is formed on the instrument 11, e.g. a channel in the instrument 11, in which an optical fiber 24 can be inserted. Preferably a rotational and/or translational movement of the light receiving device 24 relative to the electrode 15 is limited by means of the apparatus 10 such that the distal end of electrode 15 and/or light appearances created by supply of electrode 15 with electrical radio frequency energy during use of the instrument 11, always remain within the light acceptance angle 47 of the light receiving device 24. Preferably the form of a first mount 25 of apparatus 10 is adapted to the form of the instrument 11 in order to define the relative position and orientation of the light receiving device 24 relative to the electrode 15, except for such relative positions and orientations such that the tip or the distal end 50 of electrode 15 and/or the light appearances created by supply of electrode 15 with electrical radio frequency energy during use of the instrument 11 are in any remaining position within the light acceptance angle 47 of the light receiving device 24.

List of Reference Signs:

| | |
|---|---|
| 10 | apparatus |
| 11 | instrument/applicator |
| 12 | handle |
| 13 | operating element |
| 14 | electrode holding shank |
| 15 | electrode |
| 15a | working section |
| 16a | bottom side of electrode holding shank |
| 16b | top side of electrode holding shank |
| 17 | distal section |
| 18 | proximal end section/half shell |
| 19 | longitudinal side |
| 20 | top side |
| 21 | proximal section |
| 22 | transition section |
| 24 | light receiving device/fiber optic |
| 25 | first mount/first mounting channel |
| 26 | form-fit section |
| 27 | second mount |
| 28 | line |
| 28a | opening (mouth) of line |
| 29 | distal end of device |
| 30 | wall surface |
| 31 | light inlet |
| 32 | distal end of line |
| 33 | line section |
| 34 | end section of optical fiber |
| 35 | mounting bore |
| 36 | holding elements |
| 37 | inner wall surface |
| 38 | distal end of optical fiber |
| 39 | support section |
| 40 | mounting wall |
| 41 | bend |
| 42 | mounting area |
| 44 | region |
| 45a, b | side wall section |
| 46 | bottom wall section |
| 47 | light acceptance angle |
| 50a | tip of electrode |
| 50b | flat side or spatula side of electrode |
| 50c | flat side or spatula side of electrode |
| 50d | narrow side or edge of electrode |
| 50e | narrow side or edge of electrode |
| 51 | first stop |
| 52 | wall section |
| 53 | first counter stop |
| 54 | second stop |
| 55 | second counter stop |
| 56 | alignment structure |
| 57 | counter alignment structure |
| 58 | channel section |
| 58a | mouth of channel section/flushing channel |
| 59 | trocar |
| 60 | abdominal cavity |
| 61 | suction unit |
| 61a | proximal section |
| 61b | distal section |
| 61c | extension section |
| 62 | slide section |
| 63 | suction channel |
| 63a | suction opening |
| 64 | suction line |
| 65 | coupling element |
| 66 | form-fit structure |
| 67 | mount |
| 68 | supply line |
| 70 | extension element |
| 71 | line part |
| B | width |
| L | longitudinal axis |
| OA | optical axis |
| MA | center axis |
| MAV | center axis of distal section |
| ELA | electrode longitudinal axis |
| LK | light acceptance angle |
| W1 | angle |
| W2 | angle between longitudinal axis of optical fiber and first mount/ |
| | electrode |
| P | arrow |
| PP | arrow |
| PF | arrow |
| PS | arrow |
| R | axial direction |

1. An apparatus (10) configured for attachment of a light receiving device (24) for light analysis on a surgical instrument (11) or on an instrument component (12) of the surgical instrument (11) by a surgical user of the surgical instrument (11).

2. The apparatus (10) according to claim 1, wherein the apparatus (10) is configured to allow attachment of the light receiving device (24) in one single spatial position or in a plurality of predefined spatial positions relative to the instrument component (12) and/or the instrument (11).

3. The apparatus (10) according to claim 1, wherein the instrument (11) comprises an electrode (15) configured to be supplied with electrical RF energy, wherein the apparatus (10) is configured to allow attachment of the light receiving device (24) in one single spatial position or in a plurality of predefined spatial positions relative to the electrode (15).

4. The apparatus (10) according to claim 1, wherein the apparatus (10) is configured to support an electrode (15) and is configured to allow attachment of the light receiving device (24) in one single spatial position or in a plurality of predefined spatial positions relative to the electrode (15).

5. The apparatus (10) according to claim 1, comprising a mount (25) configured to limit movement of the instrument in the mount (25), except for movement of the instrument in an axial direction (L), wherein the mount (25) is configured to hold the instrument (11) in the axial direction (L) and includes a device (17, 51, 53, 21, 54, 55) for subsequent definition or limitation of the axial position of the instrument (11) in the mount (25).

6. The apparatus (10) according to claim 5, wherein the device (17, 51, 53, 21, 54, 55) for subsequent definition or limitation of the axial position of the instrument (11) comprises a section (21) that is movable transverse to the axial direction (L), wherein the section (21) is configured to engage the instrument (11) by a lateral movement of the section (21) after arrangement of the instrument (11) in the mount (25) in order to define or limit the axial position of the instrument (11) in the mount (25).

7. The apparatus (10) according to claim 6, wherein the section (21) is attached to the mount (25) in a flexible manner.

8. The apparatus (10) according to claim 1, wherein the apparatus (10) comprises a flushing channel (28, 58), wherein the flushing channel (28, 58) is configured to output a flushing medium that laterally passes a light inlet (31) of the light receiving device (24).

9. The apparatus (10) according to claim 1, wherein the apparatus (10) comprises a flushing channel (28, 58), wherein the apparatus defines a spatial position of the flushing channel relative to the light receiving device (24).

10. The apparatus (10) according to claim 8, wherein the light inlet (31) of the light receiving device (24) is arranged inside the apparatus (10) when the light receiving device (24) is attached thereto.

11. The apparatus (10) according to claim 8, wherein the light inlet (31) is arranged inside the flushing channel (58) when the light receiving device (24) is attached to the apparatus (10).

12. The apparatus (10) according to claim 8, wherein the light receiving device (24) extends at least completely or partly inside the flushing channel (28) when the light receiving device (24) is attached to the apparatus (10).

13. The apparatus (10) according to claim 1, further comprising a channel section (58) limited on one side by a wall (45a, 45b, 46) and is configured to be limited on another side by a first longitudinal side (16a) of an electrode (15) wherein the channel section (58) is configured to be flushed with a flushing medium, wherein the channel section 58 is configured such that a second longitudinal side (16b) of electrode (15) opposite the first longitudinal side (16a) is exposed at least in sections along the channel section (58) when the light receiving device (24) is attached to the apparatus (10).

14. The apparatus (10) according to claim 1, wherein an optical axis (OA) of the light receiving device (24) is oriented at an acute angle (W) with respect to a longitudinal axis of an electrode (15) of the instrument (11) when the instrument (11) is attached to the apparatus.

15. A method of using the apparatus (10) of claim 1, comprising attaching a light receiving device (24) on a surgical instrument (11) or an instrument component (12) of the surgical instrument (11) during a surgery with the apparatus (10).
